(19) United States
(12) Patent Application Publication
(10) Pub. No.: US 2024/0330448 A1
(43) Pub. Date: Oct. 3, 2024

(54) MEDICAL DEVICE BIO-FIREWALL

(71) Applicant: BOARD OF REGENTS OF THE UNIVERSITY OF NEBRASKA, Lincoln, NE (US)

(72) Inventors: William Owen Redwood, Omaha, NE (US); Jeff Dunn, Omaha, NE (US); Riley Hester, Omaha, NE (US); Ian Trent, Omaha, NE (US); Eric Wright, Omaha, NE (US)

(21) Appl. No.: 18/579,310

(22) PCT Filed: Jun. 1, 2022
(86) PCT No.: PCT/US2022/031822

§ 371 Date: Jan. 12, 2024

Related U.S. Application Data
(60) Provisional application No. 63/229,656, filed on Aug. 5, 2021.

(51) Int. Cl. G06F 21/55; G16H 40/63; G16H 40/67

(52) U.S. Cl. CPC G06F 21/554; G16H 40/63; G16H 40/67

(57) ABSTRACT

A system for providing a bio-firewall for a medical device. The system includes a bio-firewall electronic processor configured to receive a processed network command from a device electronic processor. The bio-firewall electronic processor is also configured to determine whether the processed network command is associated with a cyberattack based on at least one rule. The bio-firewall electronic processor is also configured to, in response to determining that the processed network command is not associated with the cyberattack, enable transmission, via a non-networked communication interface, of the processed network command to a non-networked component. The bio-firewall electronic processor is also configured to, in response to determining that the processed network command is associated with a cyberattack, prevent transmission, via the non-networked communication interface, of the processed network command to the non-networked component.

### CROSS-REFERENCE TO RELATED APPLICATIONS

This application claims priority to U.S. Provisional Application No. 63/229,656 filed on Aug. 5, 2021, which is incorporated fully herein by reference.

### GOVERNMENT FUNDING

The subject matter of this invention was made with Government support under contract FA8750-16-C-0178, subcontract PO-0017642 awarded by Defense Advanced Research Projects Agency (DARPA)/Air Force Research Labs (AFRL). The Government has certain rights to this invention.

### FIELD

Embodiments described herein relate to a medical device bio-firewall and, more particularly, a medical device bio-firewall for preventing performance of network command(s) suspected of being malicious.

### BACKGROUND

Biomedical devices, such as pacemakers, insulin pumps, health monitors, cardiac defibrillators, spinal cord neurostimulators, transcutaneous electrical nerve simulators, and the like, are generally configured to monitor a health condition of an associated user (or patient), perform an operation associated with a health condition of the associated user, or a combination thereof. These biomedical devices may be controlled remotely. Therefore, these biomedical devices may be subject to various cyberattacks.

### SUMMARY

Embodiments described herein relate to methods and systems of preventing medical devices from performing received network commands that are suspected of being malicious (for example, part of a cyberattack) and which possibly can adversely affect a patient using the medical device. Embodiments may be implemented using a rule-based filtering approach to log and monitor the non-networked input/output (“I/O”) of a medical device. The non-networked I/O of a medical device relates to the functioning of the medical device, for example, input to and output from patient vital sensors and actuators to control patient pacing, dosing, treatment, and the like. Traditional firewall techniques do not extend to such a non-networked I/O. By firewalling the non-networked I/O, the embodiments described herein mitigate an impact a cyberattack has on patient pacing, dosing, treatment, or the like provided by the medical device.

As one example scenario, a malicious actor (or an unauthorized user) may attempt to exploit a vulnerability in an implantable cardio defibrillator (“ICD”) by transmitting one or more malicious network commands (as part of a cyberattack against the ICD). In response to receiving the malicious network commands, the embodiments described herein determine that the network commands sent to the ICD contain parameters that, if implemented, would be outside the normal or safe bounds for the use of that particular device or patient and blocks the network commands. Additionally, in some embodiments, a cyberattack warning or alert is issued notifying the patient, a medical professional, or the like of the detected event.

Embodiments may be implemented in hardware, software, or a combination thereof. When implemented in software, the bio-firewall may be located on a medical device at the firmware or kernel level. When implemented in hardware, the bio-firewall may be deployed, for example, on a controller of the medical device between the central processing unit (“CPU”) and the I/O peripherals or on a device that sits between the medical device and an application or a device designed specifically to interface with the application programming interface (“API”) of the bio-firewall.

Accordingly, embodiments described herein provide systems and methods for providing a bio-firewall for medical devices. For example, one embodiment provides a system for providing a bio-firewall for a medical device. The system includes a bio-firewall electronic processor configured to receive a processed network command from a device electronic processor. The bio-firewall electronic processor is also configured to determine whether the processed network command is associated with a cyberattack based on at least one rule. The bio-firewall electronic processor is also configured to, in response to determining that the processed network command is not associated with the cyberattack, enable transmission, via a non-networked communication interface, of the processed network command to a non-networked component. The bio-firewall electronic processor is also configured to, in response to determining that the processed network command is associated with a cyberattack, prevent transmission, via the non-networked communication interface, of the processed network command to the non-networked component.

Another embodiment provides a method for providing a bio-firewall for a medical device. The method includes receiving, at a bio-firewall electronic processor from a device electronic processor, a processed network command. The method also includes determining, with the bio-firewall electronic processor, whether the processed network command is associated with a cyberattack based on at least one rule. The method also includes, in response to determining that the processed network command is not associated with the cyberattack, enabling, with the bio-firewall electronic processor, transmission, via a non-networked communication interface, of the processed network command to a non-networked component. The method also includes, in response to determining that the processed network command is associated with a cyberattack, preventing, with the bio-firewall electronic processor, transmission, via the non-networked communication interface, of the processed network command to the non-networked component.

Yet another embodiment provides a medical device having a bio-firewall. The medical device includes a device electronic processor configured to receive, via a networked communication interface, a network command from a user device external to the medical device. The device electronic processor is also configured to process the network command. The medical device also includes a bio-firewall electronic processor communicatively coupled to the device electronic processor. The bio-firewall electronic processor is configured to receive the processed network command from the device electronic processor. The bio-firewall electronic processor is also configured to determine whether the processed network command is associated with a cyberattack based on at least one rule. The bio-firewall electronic processor is also configured to, in response to determining that the processed network command is not associated with the cyberattack, enable transmission, via a non-networked communication interface, of the processed network command to a non-networked component. The bio-firewall electronic processor is also configured to, in response to determining that the processed network command is associated with a cyberattack, prevent transmission, via the non-networked communication interface, of the processed network command to the non-networked component.

Other aspects of the embodiments will become apparent by consideration of the detailed description and accompanying drawings.

### BRIEF DESCRIPTION OF THE DRAWINGS

FIG. 1 illustrates a system for providing a bio-firewall for medical devices according to some embodiments.

FIG. 2 illustrates a medical device included in the system of FIG. 1 according to some embodiments.

FIG. 3A illustrates a device controller included in the medical device of FIG. 1 according to some embodiments.

FIG. 3B illustrates a bio-firewall controller included in the medical device of FIG. 2 according to some embodiments.

FIG. 4 schematically illustrates an example communication flow between hardware components of the medical device according to some embodiments.

FIG. 5 illustrates a device controller included in the medical device of FIG. 1 when the functionality of the bio-firewall is implemented in software according to some embodiments.

FIG. 6 illustrates an example software implementation of the bio-firewall controller functionality during a cyberattack according to some embodiments.

FIG. 7 is a flowchart illustrating a method for providing a bio-firewall for medical devices using the system of FIG. 1 according to some embodiments.

Other aspects of the embodiments described herein will become apparent by consideration of the detailed description.

### DETAILED DESCRIPTION

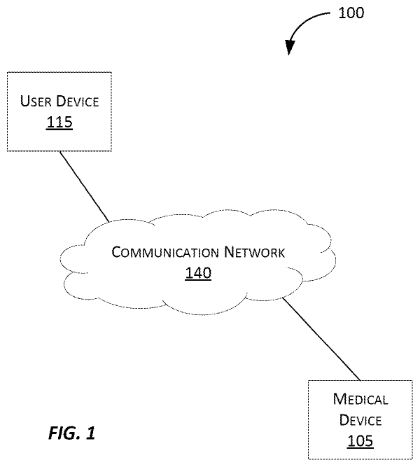

FIG. 1 schematically illustrates a system 100 for providing a bio-firewall for medical devices according to some embodiments. The system 100 includes a medical device 105 and a user device 115. In some embodiments, the system 100 includes fewer, additional, or different components than illustrated in FIG. 1. For example, the system 100 may include multiple medical devices 105, user devices 115, or a combination thereof. Additionally, in some embodiments, one or more components of the system 100 may be distributed among multiple devices, servers, or databases, combined into a single device, server, or database.

The medical device 105 and the user device 115 communicate over one or more wired or wireless communication networks 140. Portions of the communication network 140 may be implemented using a wide area network (“WAN”), such as the Internet, a local area network (“LAN”), such as a Bluetooth™ network or Wi-Fi, and combinations or derivatives thereof. Alternatively or in addition, in some embodiments, components of the system 100 communicate directly as compared to through the communication network 140. Also, in some embodiments, the components of the system 100 communicate through one or more intermediary devices not illustrated in FIG. 1.

The medical device 105 is configured to monitor a health condition of an associated user (or patient), perform an operation associated with a health condition of the associated user, or a combination thereof. The medical device 105 may be, for example, a pacemaker, an insulin pump, a health monitor, a cardiac defibrillator, a spinal cord neurostimulator, a transcutaneous electrical nerve simulator, and the like. Accordingly, in some embodiments, the medical device 105 is implanted within a user's body. However, in other embodiments, the medical device 105 is external to the user's body, such as a wearable medical device, a piece of hospital equipment, or the like.

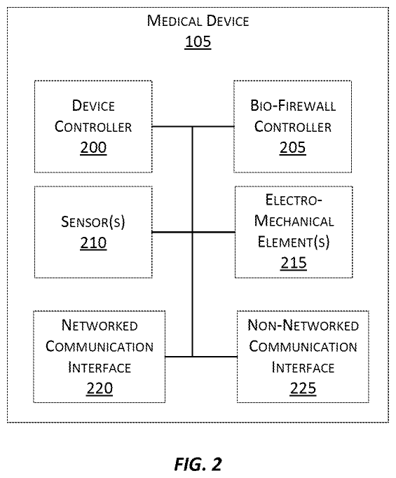

As illustrated in FIG. 2, the medical device 105 includes a device controller 200, a bio-firewall controller 205, one or more sensors 210 (referred to collectively as “the sensors 210” and individually as “the sensor 210”), one or more electro-mechanical (“EM”) elements 215 (referred to collectively as “the EM elements 215” and individually as “the EM element 215”), a networked communication interface 220, and a non-networked communication interface 225. The device controller 200, the bio-firewall controller 205, the sensor(s) 210, the EM element(s) 215, the networked communication interface 220, and the non-networked communication interface 225 communicate wirelessly, over one or more communication lines or buses, or a combination thereof. The medical device 105 may include additional, fewer, or different components than those illustrated in FIG. 2 in various configurations. As one example, the medical device 105 may include one or more energy sources configured to power the medical device 105 (or the components thereof). The medical device 105 may also perform additional functionality other than the functionality described herein. Also, the functionality (or a portion thereof) described herein as being performed by the medical device 105 may be distributed among multiple devices, such as multiple networked or communicatively coupled medical devices 105.

The sensor 210 and the EM elements 215 may be considered “non-networked” components of the medical device 105. The sensor 210 collects data related to a health condition of the user (for example, a medical device dataset). The sensor 210 may include, for example, a force sensor, a strain sensor, an image sensor, a vibration sensor, a photo optic sensor, a piezoelectric sensor, a pressure sensor, a position sensor, a temperature sensor, a blood glucose sensor, an electrocardiogram (“ECG”) sensor, a motion sensor, an inertial sensor, and the like. The data collected by the sensor 210 may be stored in a memory (not shown) of the medical device 105 (for example, a memory of the device controller 200, the bio-firewall controller 205, or the like). The EM element 215 is configured to perform an action or operation related to a health condition of a user (for example, causing a kinetic impact to the user). The EM element 215 may include, for example, a valve, an actuator, a pulse generator, an electrode, a reservoir, a motor, a pump, or the like. As one example, the EM element 215 may administer a dose of medicine. As another example, when the medical device 105 is a cardiac defibrator, the EM element 215 may generate an electric shock.

In some embodiments, the bio-firewall of the medical device 105 is implemented in software rather than in hardware like in the embodiment illustrated in FIG. 2. In some embodiments where the bio-firewall is implemented in software, the medical device 105 does not include the bio-firewall controller 205 but includes every other component illustrated in FIG. 2.

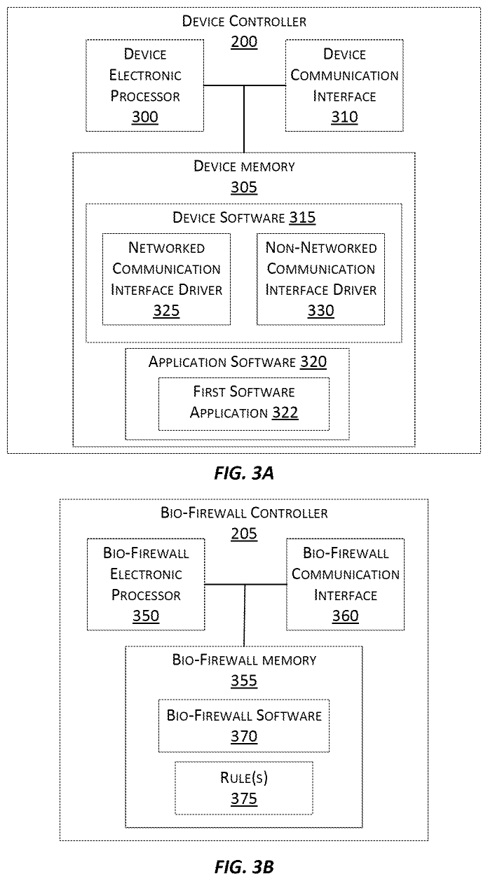

As seen in FIG. 3A, the device controller 200 includes a device electronic processor 300, a device memory 305, and a device communication interface 310. The device electronic processor 300, the device memory 305, and the device communication interface 310 communicate wirelessly, over one or more communication lines or buses, or a combination thereof. The device controller 200 may include additional, fewer, or different components than those illustrated in FIG. 3A in various configurations. The device controller 200 may also perform additional functionality other than the functionality described herein. Also, the functionality (or a portion thereof) described herein as being performed by the device controller 200 may be distributed among multiple components, such as multiple controllers.

The device electronic processor 300 includes a microprocessor, an application-specific integrated circuit (“ASIC”), or another suitable electronic device for processing data. The device memory 305 includes a non-transitory computer readable medium, such as read-only memory (“ROM”), random access memory (“RAM”) (for example, dynamic RAM (“DRAM”), synchronous DRAM (“SDRAM”), and the like), electrically erasable programmable read-only memory (“EEPROM”), flash memory, a hard disk, a secure digital (“SD”) card, another suitable memory device, or a combination thereof. The device electronic processor 300 is configured to access and execute computer-readable instructions (“software” or “code”) stored in the device memory 305. The software may include firmware, one or more applications, program data, filters, rules, one or more program modules, and other executable instructions. For example, the software may include instructions and associated data for performing a set of functions, including the methods described herein.

For example, as illustrated in FIG. 3A, the device memory 305 may store device software 315 and application software 320. In some embodiments, the application software 320 includes one or more software applications (for example, a first software application 322). In some embodiments, the first software application 322 may include software code or computer executable instructions that allow the medical device 105 to interact with a software application included on the user device 115. In some embodiment, the device software 315 includes operating system and driver code (for example, a networked communication interface driver 325 and a non-networked communication interface driver 330) and is executable by the device electronic processor 300. In some embodiments divers are software that allow the device electronic processor 300, bio-firewall electronic processor 350, or both to communicate with the user device 115, the sensor(s) 210, the electro-mechanical element(s) 215, or a combination of the foregoing via the networked communication interface 220 or non-networked communication interface 225. In some embodiments, the networked communication interface driver 325 and the first software application 322 process a received network communication and the processed network communication is analyzed by bio-firewall software 370 described below. As described in more detail below, the device software 315 operates at a more privileged level than the application software 320 and, when executed by the device electronic processor 300, controls operation of the medical device 105. For example, in some embodiments, the device software 315, when executed by the device electronic processor 300, monitors a health condition of an associated user, performs an operation associated with a health condition of an associated user, or a combination thereof. In some embodiments, the device controller 200 (via the device electronic processor 300 executing the device software 315) controls the EM elements 215 based on data collected by the sensor 210. As one example, the device electronic processor 300 may access the collected data (for example, from the device memory 305) and determine whether to perform an action or operation based on the collected data. The device electronic processor 300 may determine what action or operation to perform, how to perform that action or operation based on the collected data, or a combination thereof. Alternatively or in addition, the device electronic processor 300 controls the EM elements 215 based on network commands or signals, such as a network command received from the user device 115. A network command may include, for example, an operating parameter for the medical device 105, an alert or alarm setting, a dosage setting, a dosage schedule, a testing schedule, an operating parameter range, or the like. As one example, the device electronic processor 300 may receive a network command establishing a medicine dosing schedule. Using the medicine dosing schedule, the device electronic processor 300 may control the EM element 215 such that the EM element 215 administers medicine according to the received medicine dosing schedule. Accordingly, in some embodiments, the device electronic processor 300 may transmit one or more control signals to a corresponding EM element 215. In response to receiving the control signal(s), the EM element 215 may perform the action or operation according to the control signal(s).

The device communication interface 310 allows the device controller 200 to communicate with devices external to the device controller 200. For example, as illustrated in FIG. 2, the device controller 200 may communicate with the bio-firewall controller 205, the sensor(s) 210, the EM element(s) 215, the networked communication interface 220, the non-networked communication interface 225, or a combination thereof through the device communication interface 310. In particular, the device communication interface 310 may include a port for receiving a wired connection to an external device (for example, a universal serial bus (“USB”) cable and the like), a transceiver for establishing a wireless connection to an external device (for example, over one or more communication networks 140, such as the Internet, a LAN, a WAN, and the like), or a combination thereof.

As seen in FIG. 3B, the bio-firewall controller 205 includes a bio-firewall electronic processor 350 (for example, a microprocessor, an ASIC, or another suitable electronic device for processing data), a bio-firewall memory 355 (for example, a non-transitory computer-readable medium), and a bio-firewall communication interface 360. The bio-firewall electronic processor 350, the bio-firewall memory 355, and the bio-firewall communication interface 360 communicate wirelessly, over one or more communication lines or buses, or a combination thereof. The bio-firewall controller 205 may include additional, fewer, or different components than those illustrated in FIG. 3B in various configurations. The bio-firewall controller 205 may also perform additional functionality other than the functionality described herein. Also, the functionality (or a portion thereof) described herein as being performed by the bio-firewall controller 205 may be distributed among multiple components, such as multiple controllers. Alternatively or in addition, in some embodiments, the functionality (or a portion thereof) described herein as being performed by the bio-firewall controller 205 may be combined with another device or controller, such as the device controller 200.

The bio-firewall electronic processor 350 is configured to access and execute computer-readable instructions (“software”) stored in the bio-firewall memory 355. The software may include firmware, one or more applications, program data, filters, rules, one or more program modules, and other executable instructions. For example, the software may include instructions and associated data for performing a set of functions, including the methods described herein.

For example, as illustrated in FIG. 3B, the bio-firewall memory 355 may store bio-firewall software 370 and one or more rules 375 (referred to herein collectively as “the rules 375” and individually as “the rule 375”). The bio-firewall software 370 is a software application executable by the bio-firewall electronic processor 350. A rule 375 may include, for example, a programming rule or model that defines what is deemed “safe” for inputs and outputs (for example, network commands) of the device controller 200. As described in more detail below, the bio-firewall software 370, when executed by the bio-firewall electronic processor 350, filters analog inputs and outputs (for example, network commands) of the device controller 200, determines safe inputs and outputs (for example, network commands) of the device controller 200 based on one or more rules 375, and allows safe inputs and outputs (for example, network commands) of the device controller 200. As one example, when the medical device 105 is an implantable cardio defibrillator (“ICD”), a rule 375 may define a safe operating range. When an input or output (for example, network commands) of the device controller 200 is outside of the safe operating range, the bio-firewall software 370 (using the rule 375) may determine that input or output (for example, a network command) as not “safe,” such that the bio-firewall software 370 ultimately blocks or prevents that input or output. As another example, when the medical device 105 is an insulin pump, a rule 375 may define a safe amount of insulin to deliver in a predetermined period of time to prevent an attacker from sending a large number of small doses of insulin in a short enough period of time to be harmful to the patient. Accordingly, in some embodiments, the bio-firewall controller 205 (via execution of the bio-firewall software 370 by the bio-firewall electronic processor 350) functions as a firewall that mitigates kinetic impacts that may be caused by a cyberattack without modifying the device software 315 of the device controller 200.

In some embodiments, the bio-firewall software 370 logs, in a device log, an administered amount and a time when the amount is administered. For example, when the medical device 105 is an insulin pump, the bio-firewall software 370 may record, in a device log stored in the device memory 305 or the bio-firewall memory 355, the amount of insulin administered in a dose and a time when the does is administered. In some embodiments, the bio-firewall software 370 may use a device log to determine whether an input or output (for example, a network command) violates a rule 375. In some embodiments, the bio-firewall software 370 also logs sensor data from the sensor 210.

The bio-firewall communication interface 360 allows the bio-firewall controller 205 to communicate with devices external to the bio-firewall controller 205. For example, as illustrated in FIG. 2, the bio-firewall controller 205 may communicate with the device controller 200, the sensor(s) 210, the EM element(s) 215, the networked communication interface 220, the non-networked communication interface 225, or a combination thereof through the bio-firewall communication interface 360. In particular, the bio-firewall communication interface 360 may include a port for receiving a wired connection to an external device (for example, a universal serial bus (“USB”) cable and the like), a transceiver for establishing a wireless connection to an external device (for example, over one or more communication networks 140, such as the Internet, a LAN, a WAN, and the like), or a combination thereof.

Returning to FIG. 2, the medical device 105 also includes the networked communication interface 220 and the non-networked communication interface 225. The networked communication interface 220 allows the medical device 105 to communicate with devices, components, or a combination thereof external to the medical device 105. As one example, the networked communication interface 220 allows the medical device 105 to communicate (via the communication network 140) with “networked” devices, such as the user device 115. The non-networked communication interface 225 allows communication with non-networked components or devices of the medical device 105. As one example, the non-networked communication interface 225 allows communication between the bio-firewall controller 205 and one or more non-networked components, such as the sensor(s) 210, the EM element(s) 215, or a combination thereof. In particular, the networked communication interface 220, the non-networked communication interface 225, or a combination thereof may include a port for receiving a wired connection to a device (for example, a universal serial bus (“USB”) cable and the like), a transceiver for establishing a wireless connection to a device (for example, over one or more communication networks 140, such as the Internet, a LAN, a WAN, and the like), or a combination thereof. In some embodiments, the networked communication interface 220 and the non-networked communication interface 225 are combined into a single communication interface.

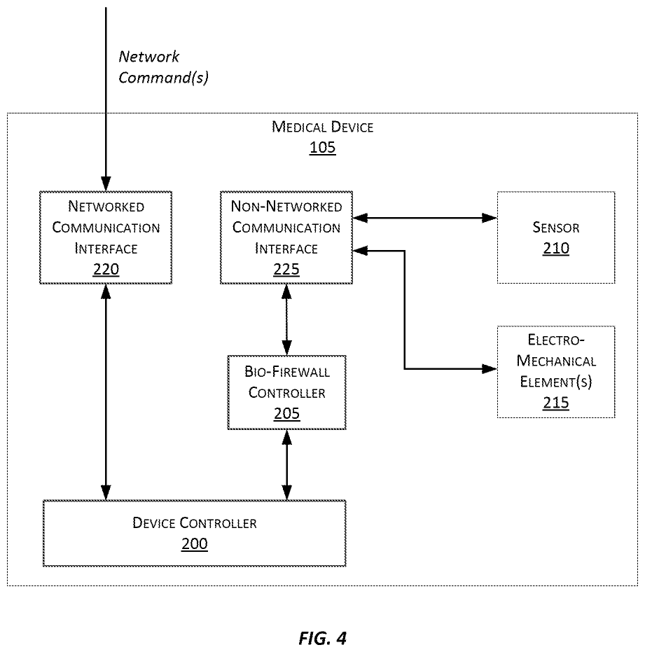

FIG. 4 schematically illustrates an example communication flow between hardware components of the medical device 105 when the bio-firewall is implemented in hardware according to some embodiments. In the illustrated example of FIG. 4, network commands (from, for example, the user device 115) are received via the networked communication interface 220 of the medical device 105. The received network command is provided to the device controller 200 for processing (via, for example, the networked communication interface driver 325 and the first software application 322 executed by the device electronic processor 300). The processed network command (for example, as analog outputs) of the device controller 200 is passed to the bio-firewall controller 205 for firewall filtering (via, for example, the bio-firewall software 370 executed by the bio-firewall electronic processor 350). Allowed network commands are transmitted to non-networked component(s) (for example, the sensor(s) 210, the EM element(s) 215, or a combination thereof) of the medical device 105 via the non-networked communication interface 225. In response to receiving the allowed network command(s), the non-networked component(s) (for example, the sensor(s) 210, the EM element(s) 215, or a combination thereof) of the medical device 105 perform an action or operation accordingly.

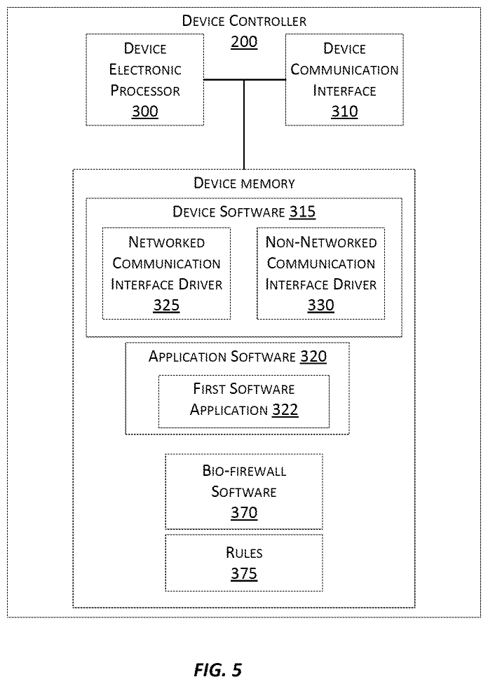

As described above, in some embodiments, the functionality of the bio-firewall controller 205 (for example, the bio-firewall software 370) may be provided as a software implementation via the device controller 200 (as opposed to a hardware implementation via the bio-firewall controller 205). As illustrated in FIG. 5, when the bio-firewall is implemented in software, the device memory 305 of the device controller 200 may include the bio-firewall software 370, the rule(s) 375, or a combination thereof in addition to the device software 315 and application software 320. With reference to FIG. 5, in such embodiments, the bio-firewall software 370, the rule(s) 375, or a combination thereof is stored in a secure storage (for example, the device memory 305). In some embodiments, the bio-firewall software 370, the rule(s) 375, or a combination thereof are included in the operating system of the medical device 105.

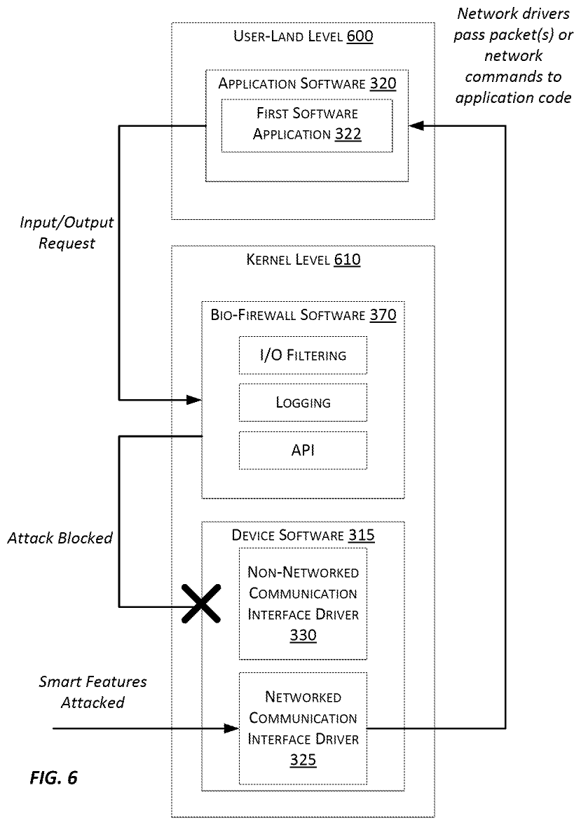

FIG. 6 schematically illustrates an example communication flow between software components of the medical device 105 when the bio-firewall is implemented in software according to some embodiments. According to such embodiments, the bio-firewall software 370, the rule(s) 375, or a combination thereof may be at a firmware level on the medical device 105 and provide introspection into the cyber health the medical device 105 and allow for device log retrieval, I/O blocking, and alerting through a standardized API to a smart device application or other device designed to interface with the API, as seen in FIG. 6.

As illustrated in FIG. 6 there are two levels of software included in the medical device 105. The first level is user-land level 600 and the user-land level 600 includes the application software 320. The second level is the kernel level 610. The kernel level 610 is a more privileged level than the user-land level 600. The kernel level 610 includes the device software 315 (including, for example, driver and operating system software), the bio-firewall software 370, and, the rules 375 (not illustrated). In some embodiments, software code included in the user-land level 600 cannot directly interface with the sensor(s) 210 or the electromechanical element(s) 215. In some embodiments, the software code included in the user-land level 600 may only interact with the sensor(s) 210 or the electromechanical element(s) 215 by communicating with the software code at the kernel level 610. In some embodiments, the software code included in the kernel level 610 is written by the manufacturer of the medical device 105 and allows the medical device 105 to function at a basic level (for example, if the medical device 105 is a pacemaker, the kernel level 610 software code allows the medical device 105 to receive input from the sensor 210 and administer a shock based on the sensor input). In some embodiments the software code included in the user-land level 600 is written by one or more software vendors and allows the medical device 105 to communicate with an application included in, for example, a smart phone belonging to the patient with the medical device 105.

As illustrated in FIG. 6, when the bio-firewall is implemented in software, a processed network command that is associated with (or potentially associated with) the cyberattack is prevented (or blocked) by the device electronic processor 300 from being transmitted or sent to the non-networked communication interface driver 330 and, ultimately, the non-networked component via the non-networked communication interface 225.

Returning to FIG. 1, the user device 115 is a computing device and may include, for example, a desktop computer, a terminal, a workstation, a laptop computer, a tablet computer, a smart watch or other wearable, a smart television or whiteboard, or the like. Alternatively or in addition, the user device 115 may be a mobile communication device, such as a smart cellular device or phone. Although not illustrated, the user device 115 may include similar components as the device controller 200 (an electronic processor, a memory, and a communication interface). In some embodiments, the user device 115 may also include a human-machine interface for interacting with a user. The human-machine interface may include one or more input devices, one or more output devices, or a combination thereof. Accordingly, in some embodiments, the human-machine interface allows a user to interact with (for example, provide input to and receive output from) the user device 115. For example, the human-machine interface may include a keyboard, a cursor-control device (for example, a mouse), a touch screen, a scroll ball, a mechanical button, a display device (for example, a liquid crystal display (“LCD”)), a printer, a speaker, a microphone, or a combination thereof. In some embodiments, the human-machine interface includes a display device. The display device may be included in the same housing as the user device 115 or may communicate with the user device 115 over one or more wired or wireless connections. For example, in some embodiments, the display device is a touchscreen included in a laptop computer, a tablet computer, or a mobile communication device. In other embodiments, the display device is a monitor, a television, or a projector coupled to a terminal, desktop computer, or the like via one or more cables.

A user of the user device 115 may include, for example, an unauthorized user, a malicious user, or an attacker. In other words, an unauthorized user may interact or interface with the medical device 105 using the user device 115 for malicious reasons. As one example, an unauthorized user may discover a vulnerability with respect to the medical device 105 and attempt to exploit that vulnerability against the patient (for example, using the user device 115). The unauthorized user may utilize an attack to exploit the vulnerability and cause a kinetic impact on the patient associated with the medical device 105.

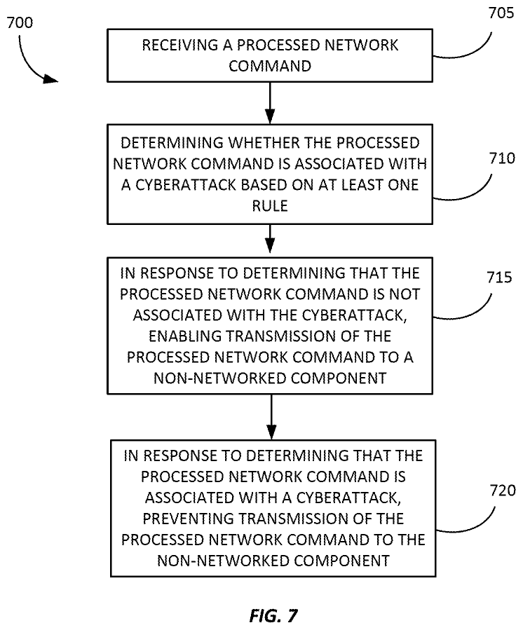

FIG. 7 illustrates a method 700 for providing a bio-firewall for medical devices using the system 100 of FIG. 1 according to some embodiments. The method 700 is described herein as being performed by the bio-firewall controller 205 of the medical device 105 and, in particular, the bio-firewall software 370 as executed by the bio-firewall electronic processor 350. However, as noted above, the functionality performed by the medical device 105 (or a portion thereof) may be performed by other devices, such as, for example, the device controller 200.

As illustrated in FIG. 7, the method 700 includes receiving a processed network command (at block 705). As noted above with respect to FIG. 4, network commands (from, for example, the user device 115) are received via the networked communication interface 220 of the medical device 105. The received network command is provided to the device controller 200 for processing. In some embodiments where the bio-firewall is implemented in hardware using the bio-firewall controller 205, processing the network command includes executing the networked communication interface driver 325 to determine that there is a software application (for example, the first software application 322) included in the application software 320 which is capable of receiving and using the network command. Processing the network command also includes sending the network command to the first software application 322 and the first software application 322 sending the network command to the non-networked communication interface driver 330. The non-networked communication interface driver 330 attempts to send a processed network command to a sensor 210 or an electromechanical element 215 via the non-networked communication interface 225 but the processed network command from the device controller 200 is intercepted by the bio-firewall controller 205. Accordingly, in some embodiments, the processed network command is received by the bio-firewall controller 205 (for example, the bio-firewall electronic processor 350) from the device controller 200 (for example, the device electronic processor 300) after processing of the network command is performed by the device controller 200 (for example, via the device software 315 executed by the device electronic processor 300).

In some embodiments where the bio-firewall is implemented in software, processing the network command includes executing the networked communication interface driver 325 to determine that there is a software application (for example, the first software application 322) included in the application software 320 which is capable of receiving and using the network command. Processing the network command also includes sending the network command to the first software application 322 and the first software application 322 then sending the processed network command to the bio-firewall software 370 included in the kernel level 610.

In response to receiving the processed network command (at block 705), the bio-firewall electronic processor 350 determines whether the processed network command is associated with a cyberattack based on at least one rule 375 (at block 710). As seen in FIG. 3B, the rule 375 may be stored in the bio-firewall memory 355. However, in other embodiments, the rule(s) 375 may be stored in another remote storage location or device. Accordingly, in some embodiments the bio-firewall electronic processor 350 accesses one or more rules 375 from the bio-firewall memory 355 (or another remote storage location or device). As noted above, a rule 375 may include, for example, a programming rule or model that defines what is deemed “safe” or authorized for inputs and outputs (for example, network commands) of the device controller 200.

In some embodiments, the rule 375 includes a range for an operating parameter (or an operating range) of the medical device 105. For example, the medical device 105 may be associated with specific operating parameters that are deemed “safe” for the given medical device 105 (for example, as set by a manufacturer of the medical device 105), the given patient associated with the medical device 105 (for example, as set by a medical professional or doctor treating the patient), or a combination thereof. Accordingly, in some embodiments, the bio-firewall electronic processor 350 determines whether the processed network command is associated with a cyberattack by comparing the network command to a range for an operating parameter. As one example, the medical device 105 may be associated with a range for an operating parameter in which the medical device 105 may safely operate within (a manufacturer set range). According to this example, the bio-firewall electronic processor 350 may determine that the network command is associated with (or potentially associated with) a cyberattack when the network command alters an operating parameter such that the operating parameter no longer falls within the range for that operating parameter. As another example, the medical device 105 may be associated with a range for an operating parameter based on a health condition of a patient associated with the medical device 105 (for example, a patient-specific range for an operating parameter as set by a medical professional or doctor treating the patient). According to this example, the bio-firewall electronic processor 350 may determine that the network command is associated with (or potentially associated with) a cyberattack when the network command alters an operating parameter such that the operating parameter no longer falls within the patient-specific range for that operating parameter.

In some embodiments, the bio-firewall electronic processor 350 determines whether the processed network command is associated with a cyberattack based on a rule 375 and data collected by the sensor(s) 210. For example, when the network command alters an operating parameter for the medical device 105, the bio-firewall electronic processor 350 may compare the altered operating parameter to current data provided by the sensor(s) 210. As one example, when the medical device 105 is an insulin pump and the network command alters an insulin dosage for the insulin pump, the bio-firewall electronic processor 350 may confirm the dosage based on current blood sugar data collected by the sensor(s) 210. According to this example, the bio-firewall electronic processor 350 may determine that the network command is associated with (or potentially associated with) a cyberattack when the insulin dosage is not consistent with the current blood sugar data (for example, when the insulin dosage would be too high or too low given the current blood sugar data).

In some embodiments, the bio-firewall electronic processor 350 determines whether the processed network command is associated with a cyberattack based on a rule 375 and data included in a device log. As one example, when the medical device 105 is an insulin pump and the network command requests an insulin dose to be administered the bio-firewall electronic processor 350 may confirm the dosage based on device logs. According to this example, when the network command would cause an amount of insulin delivered in a predetermined amount of time to reach or exceed a maximum amount of insulin that may be administered during the predetermined period of time, bio-firewall electronic processor 350 may determine that the network command is associated with (or potentially associated with) a cyberattack.

In response to determining that the processed network command is not associated with the cyberattack, the bio-firewall electronic processor 350 enables transmission (via the non-networked communication interface 225) of the processed network command to a non-networked component (at block 715). As noted above, a non-networked component of the medical device 105 may include, for example, one or more sensors 210, one or more EM elements 215, or a combination thereof. Accordingly, in some embodiments, the bio-firewall electronic processor 350 transmits the processed network command to a non-networked component. In response to receiving the processed network command, the non-networked component may perform an action or function in accordance with the processed network command. As one example, when the network command alters an operating parameter for an EM element 215 and the bio-firewall electronic processor 350 determines that the altered operating parameter is safe (i.e., not associated with a cyberattack), the bio-firewall electronic processor 350 transmits the altered operating parameter to the EM element 215 and the EM element 215 performs an operation using the altered operating parameter.

In response to determining that the processed network command is associated with (or potentially associated with) the cyberattack, the bio-firewall electronic processor 350 prevents (or blocks) transmission (via the non-networked communication interface 225) of the processed network command to a non-networked component (at block 720). By blocking or preventing the transmission of a potentially malicious network command, impacts of cyberattacks on patients may be mitigated or eliminated.

Additionally, in some embodiments, the bio-firewall electronic processor 350 also generates and transmits a cyberattack warning (or alert) in response to determining that the processed network command is associated with (or potentially associated with) the cyberattack. A cyberattack warning may include, for example, a visual alert, an audio alert, a tactile alert, or the like. The cyberattack warning may include, an indication of a potential cyberattack against the medical device 105 and information associated with the potential cyberattack. Information associated with the potential cyberattack may include, for example, a source of the network command triggering the potential cyberattack (for example, log-in information, GeoIP information, device identifying information, or the like), the network command (for example, the altered operating parameter), a rule 375 triggering the cyberattack warning (for example, the rule 375 that the bio-firewall electronic processor 350 used to determine or detect the potential cyberattack), a reasoning for the cyberattack warning (for example, a brief description of why the network command triggered the cyberattack warning), or the like.

In some embodiments, the bio-firewall electronic processor 350 may transmit the cyberattack warning to a human-machine interface associated with the medical device 105 (not illustrated). The human-machine interface associated with the medical device 105 may include, for example, a visual indicator light (for example, an LED), a speaker, a motor, a display device (for example, an LCD), or the like. The human-machine interface associated with the medical device 105 may be included in the housing of the medical device 105, external to the housing of the medical device 105, or a combination thereof. In some embodiments, when the medical device 105 is implanted within a patient, the human-machine interface is external to the housing of the medical device 105. For example, the human-machine interface may be included in a user device (similar to the user device 115) that is in communication with the medical device 105 and belongs to a patient, healthcare personnel, or cybersecurity personnel. In other embodiments, when the medical device 105 is not implanted within a patient the human-machine interface may be included within the housing of the medical device 105, external to the housing of the medical device 105, or a combination thereof.

It should be understood that while blocks 710, 715, and 720 are described above as being performed by the bio-firewall electronic processor 350, in some embodiments, blocks 710, 715, and 720 are only performed by the bio-firewall electronic processor 350 in hardware implementations of a bio-firewall. When the bio-firewall is implemented in blocks 710, 715, and 720 may be performed by the device electronic processor 300 when the device electronic processor 300 executes the bio-firewall software 370 and rules 375 included in the device memory 305 of the device controller 200 illustrated in FIG. 5. Additionally, in some embodiments, the device electronic processor 300, rather than the bio-firewall electronic processor 350, generates and transmits a cyberattack warning (or alert) in response to determining that the processed network command is associated with (or potentially associated with) the cyberattack.

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. Furthermore, some embodiments described herein may include one or more electronic processors configured to perform the described functionality by executing instructions stored in non-transitory, computer-readable medium. Similarly, embodiments described herein may be implemented as non-transitory, computer-readable medium storing instructions executable by one or more electronic processors to perform the described functionality. As used in the present application, “non-transitory computer-readable medium” comprises all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

Various features and advantages of the embodiments are set forth in the following claims.

What is claimed is:

1. A system for providing a bio-firewall for a medical device, the system comprising:
a bio-firewall electronic processor configured to
receive a processed network command from a device electronic processor,
determine whether the processed network command is associated with a cyberattack based on at least one rule,
in response to determining that the processed network command is not associated with the cyberattack,
enable transmission, via a non-networked communication interface, of the processed network command to a non-networked component, and
in response to determining that the processed network command is associated with the cyberattack,
prevent transmission, via the non-networked communication interface, of the processed network command to the non-networked component.

2. The system of claim 1, wherein the processed network command includes at least one selected from a group consisting of an operating parameter, an alert setting, a dosage setting, a dosage schedule, a testing schedule, and an operating range.

3. The system of claim 1, wherein the bio-firewall electronic processor is configured to determine whether the processed network command is associated with the cyberattack based on the at least one rule and data collected by a sensor associated with the medical device.

4. The system of claim 1, wherein the bio-firewall electronic processor is further configured to
in response to determining that the processed network command is associated with the cyberattack,
generate and transmit a cyberattack warning for display to a user of the medical device.

5. The system of claim 1, wherein the at least one rule includes a range for an operating parameter of the medical device.

6. The system of claim 1, wherein the at least one rule includes an operating parameter based on the medical device.

7. The system of claim 1, wherein the at least one rule includes an operating parameter based on a health condition of a user associated with the medical device.

8. A method of providing a bio-firewall for a medical device, the method comprising:
receiving a processed network command;
determining whether the processed network command is associated with a cyberattack based on at least one rule;
in response to determining that the processed network command is not associated with the cyberattack,
enabling transmission, via a non-networked communication interface, of the processed network command to a non-networked component; and
in response to determining that the processed network command is associated with the cyberattack,
preventing transmission, via the non-networked communication interface, of the processed network command to the non-networked component.

9. The method of claim 8, wherein receiving the processed network command includes receiving at least one selected from a group consisting of an operating parameter, an alert setting, a dosage setting, a dosage schedule, a testing schedule, and an operating range.

10. The method of claim 8, wherein determining whether the processed network command is associated with the cyberattack includes determining whether the processed network command is associated with the cyberattack based on the at least one rule and data collected by a sensor associated with the medical device.

11. The method of claim 8, wherein enabling transmission of the processed network command to the non-networked component includes enabling transmission of the processed network command to a sensor configured to collect data related to a health condition of a user of the medical device.

12. The method of claim 8, wherein enabling transmission of the processed network command to the non-networked component includes enabling transmission of the processed network command to an electro-mechanical element configured to perform an action or operation related to a health condition of a user of the medical device.

13. The method of claim 8, further comprising:
in response to determining that the processed network command is associated with the cyberattack,
generating and transmitting a cyberattack warning for display to a user of the medical device.

14. The method of claim 8, wherein determining whether the processed network command is associated with a cyberattack based on the at least one rule includes determining whether the processed network command is associated with the cyberattack based on an operating parameter associated with a health condition of a user associated with the medical device.

15. A medical device having a bio-firewall, the medical device comprising:
a device electronic processor configured to
receive, via a networked communication interface, a network command from a user device external to the medical device, and
process the network command; and
a bio-firewall electronic processor communicatively coupled to the device electronic processor, the bio-firewall electronic processor configured to
receive the processed network command from the device electronic processor,
determine whether the processed network command is associated with a cyberattack based on at least one rule,
in response to determining that the processed network command is not associated with the cyberattack,
enable transmission, via a non-networked communication interface, of the processed network command to a non-networked component, and
in response to determining that the processed network command is associated with the cyberattack,
prevent transmission, via the non-networked communication interface, of the processed network command to the non-networked component.

16. The medical device of claim 15, further comprising:
the non-networked component, wherein the non-networked component is configured to perform an operation based on the processed network command.

17. The medical device of claim 15, wherein the bio-firewall electronic processor is further configured to
in response to determining that the processed network command is associated with the cyberattack,
generate and transmit a cyberattack warning for display to a user of the medical device.

18. The medical device of claim 15, wherein the at least one rule includes a range for an operating parameter of the medical device.

19. The medical device of claim 15, wherein the non-networked component includes a sensor configured to collect data related to a health condition of a user of the medical device.

20. The medical device of claim 15, wherein the non-networked component includes an electro-mechanical element configured to perform an action or operation related to a health condition of a user of the medical device.
